(19) United States
(12) Patent Application Publication
(10) Pub. No.: US 2008/0313751 A1
(43) Pub. Date: Dec. 18, 2008

(54) Method by Using Human or Mice-Isg12 to Develop and Prepare Drugs and Single Nucleotide Polymorphism in the Isg12 Gene for Diagnostic Use

(76) Inventors: Nikolina Papac-Milicevic, Wien (AT); Pavel Uhrin, Wien (AT); Gernot Schabbauer, Tulin (AT); Elisabeth Binder, Wien (AT); Bernd Binder, Wien (AT)

Correspondence Address: K.F. ROSS P.C., 5683 RIVERDALE AVENUE, SUITE 203 BOX 900, BRONX, NY, 10471-0900, US

(21) Appl. No.: 12/092,189

(22) PCT Filed: Oct. 31, 2006
(86) PCT No.: PCT/AT2006/000448

§ 371 Date: Jun. 24, 2008

(30) Foreign Application Priority Data
Oct. 31, 2005 (AT) A 1790/2005

(51) Int. Cl. A01K 67/027; C12Q 1/68

(52) U.S. Cl. 800/3; 435/6

(57) ABSTRACT

Here is described a novel gene, the interferon inducible gene 12 (ISG12, IFI27), that interacts with nuclear receptors and enhances nuclear export of such transcription factors. As a consequence, the transcriptional activities of these nuclear receptors are decreased. As examples effects on NR4A1 and PPARa and PPARg are given. When ISG12 is absent as in ISG12 deficient mice transcriptional activities of these nuclear receptors are not impaired and their protective effects are fully appreciated. Consistently, ISG12 deficient mice are resistant to restenosis upon carotid artery ligation and to endotoxin induced death. Human genetics studies indicate the importance of ISG12 where the inventors could show a strong association between an intronic ISG12 SNP and the presence of hypercholesterolemia, diabetes type 2 and stroke. ISG12 is therefore a target for novel therapeutic strategies for the treatment of vascular diseases.

The present invention relates to the use of the interferon inducible gene 12 (ISG12, IFI27) to modulate transcriptional activities of nuclear receptors; the use of mice deficient in ISG12 as animal models for the study of inflammatory and metabolic diseases and the use of single nucleotide polymorphism in the human ISG12 gene as marker for disease susceptibility

### STATE OF THE ART

Inflammation is a major factor mediating occlusive vascular diseases and in fact atherosclerosis can be regarded as a chronic inflammatory disease^{1,2}. Consistently monocytes^{3,4}, macrophages⁵ and T_{Hl} cells⁶ are found in early and late lesions and are thought to be responsible for generating the specific cytokine environment mediating progression of the disease⁷. In addition to the nuclear factor kappa B (NFκB)⁸, the major transcription factor mediating the inflammatory response, nuclear receptors are also targets for regulation by such a mixture of cytokines. With respect to nuclear receptors the inventors and others could show that NR4A1, a member of the NR4R family of orphan nuclear receptors, is up-regulated in the inflamed vasculature, specifically in endothelium, smooth muscle cells and monocytes⁹⁻¹¹. In mice transgenic for a dominant negative form of NR4A1 a significantly higher degree of restenosis was found in a carotid artery ligation model and adenoviral over expression of wild type NR4A1 abolished the restenosis process¹². Both findings indicate a beneficial role of NR4A1 in vascular diseases. Therefore NR4A1 in concert with other nuclear receptors such as the adopted nuclear receptors PPARa and PPARg seem to be involved in feedback loops counteracting vascular pathologies¹³⁻¹⁵.

NR4A1 (Nur77) belongs to the NR4R family of orphan nuclear receptors with hitherto unknown ligands for transcriptional regulation. Although recently prostaglandin A¹⁶ was described to act as transactivator for NR4A3, the activity of members of the NR4R family is thought to be mainly dependent on regulated expression and posttranslational modification. Although activities of co-repressors¹⁷⁻¹⁹ and coactivators²⁰ were also reported, none of these regulators is known to be involved in vascular diseases. The inventors therefore searched for further factors modulating the activity of these nuclear receptors that might attenuate their beneficial effects in vascular diseases.

Parker, N. et al “Identification of a novel gene family that includes the interferon-inducible human genes 6-16 and ISG12” BMC genomics [electronic resource] 2004, vol. 5, No. 4 pubmed: 14728724 describes the silico identification of a gene family to which belongs ISG12. As stated by the authors hitherto its function is unknown and therein no indication is found relating to the possible function of ISG12 or to which ISG12 could be used.

US 2005/0009067 A1 discloses the use of the combination of a lot of genes amongst also ISG12 for “gene expression profiling” of pancreas carcinomas. “Drug screening” therein described relates to the effect of various drugs on the expression profile of the described markers in response to treatment with the respective drugs or inhibition of expression of the markers on nRNA or by—not defined—antibodies and not on the effect of ISG12 which had a hitherto unknown—if at all—effect as an aim of such drugs, particularly for modification of the activity of transcription factors. The essence of this disclosure relates to a method for characterizing of pancreas tissue, a kit for characterizing and a method for screening of pancreas cells in regard to the effect of test components to modifications of 2 or more of the above mentioned genes.

WO 2004/011618 A1 relates to a method for identification of genes which are overexpressed in fat of different mice strains as well as to the use if identified genes for inhibition of adipogenesis, for treatment of diabetes and for screening of low molecular substances which modulate adipogenesis and for treatment of diabetes respectively. One of the found gene also is ISG12 what is not surprising because in known manner ISG12 will be regulated by interferon. Therein also is disclosed a bioassay for identifying of substances which can inhibit fat collection whereby cells which express one of the identified proteins will be exposed to substances and subsequently the cells are analyzed relating to changes in its activity. Therein in no manner the activity of ISG12 relating to the modulation of the activity of transcription factors is brought up.

Labrada, L. et al “Age-dependant resistance to lethal alphavirus encephalitis in mice: analysis of gene expression in the central nervous system and identification of a novel interferon-inducible protective gene, mouse ISG12” Journal of virology, 2002, vol. 76, No. 22, pp. 11688-11703 describe the different susceptibility of newborn vis-á-vis four weeks old mice against Sindbis virus infections and describe that ISG12 is upregulated in the less susceptible four weeks old mice and that over expression of ISG12 is protective in newborn mice against Sindbis virus. The over expression of ISG12 as response to virus infection is not surprising because of the interferon-inducibility of ISG12 and the protective effect of ISG12 in no manner will be brought in connection with the modulation of the activity of transcription factors.

JP 2005204549 A discloses a method for examination of the susceptibility against the progress of a HCV infection. In population studies SNPs were found in 103 genes which correlate with the progress of a HCV infection. Likewise not surprising is one of the genes ISG12 which is upregulated at virus infections through interferons; also herein in no manner ISG12 will be brought in connection with the modulation of the activity of transcription factors.

### INVENTION

The inventors found unexpectedly a novel modulator of the activities of several nuclear receptors, the interferon regulated gene 12 (ISG12) present under basal conditions at low levels in the nuclear envelope. From data obtained in vitro, in ISG12 knock out mice in vivo and from the frequency of polymorphism in the ISG12 gene in patients, the inventors conclude that ISG12 is a novel gene upregulated in the vasculature upon injury that contributes to vascular pathologies by increasing nuclear export of “beneficial” nuclear receptors thereby down-regulating their transcriptional activities. ISG12 therefore might represent a target for novel therapeutic strategies for the treatment of vascular diseases.

Results:

Finding of ISG12:

In search for NR4A1 interacting proteins the inventors constructed a bait of a truncated NR4A1 (amino acids 248-557) and used it in a yeast two hybrid screen^{21,22}. One of the positive interaction partners found in a library made from cytokine activated microvascular endothelial cells represented the full length cDNA of the interferon stimulated gene 12 (ISG12, GI:55925613).

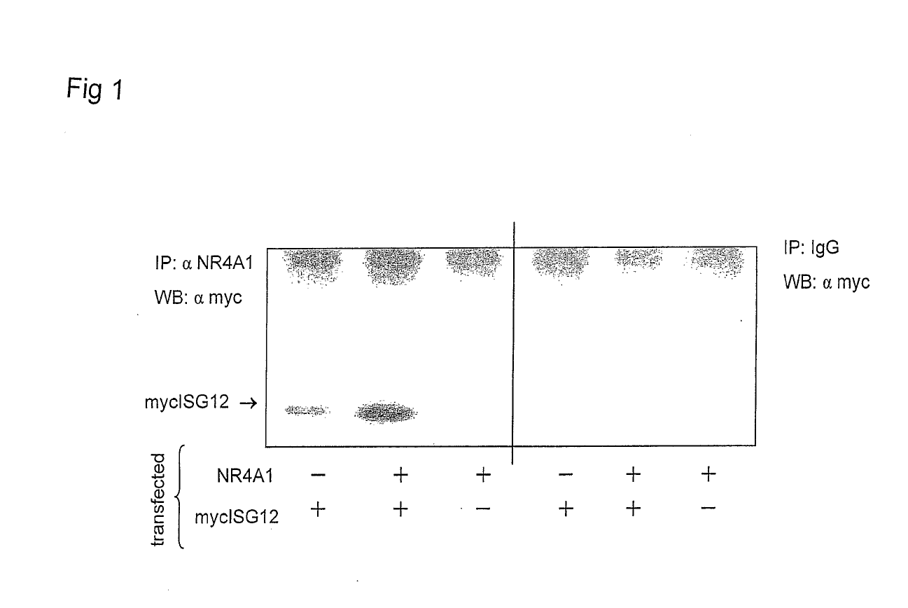

Interaction in the yeast was verified by “back transformation” (data not shown) and interaction in mammalian cells was demonstrated by co-immunoprecipitation of myc-tagged ISG12 by an antibody against NR4A1 that precipitated endogenous NR4A1 (FIG. 1a, lane 1) and over-expressed full length NR4A1 (FIG. 1 lane 2) in 293 cells. FIG. 1 shows that NR4A1 interacts with ISG12. Co-immunoprecipitation in 293 cells: NR4A1 was overexpressed in 293 cells either in the absence or the presence of ISG12. Lysates from 293 cells were immunoprecipitated for 4 hrs with anti-NR4A1 or control IgG; for immunoblotting a monoclonal mouse anti-myc antibody was used. ISG12 co-precipitates with endogenous and overexpressed NR4A1, (n=3).

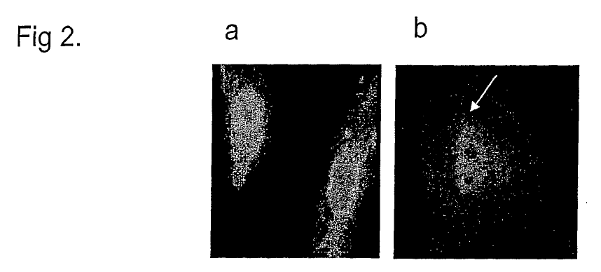

To determine the site in the cell where that interaction takes place, the inventors overexpressed myc-tagged ISG12 and EGFP-tagged NR4A1 in human umbilical vein endothelial cells (HUVECs; FIG. 2a). NR4A1 was located predominantly in the nucleus while ISG12 was located mainly around the nucleus where it co-localized with NR4A1 as revealed by the merged pictures obtained by confocal laser microscopy (yellow ring). These data are consistent with the published localization of ISG12 in the nuclear envelope²³. Reconstructing the picture from the Z-stacks of the confocal laser microscopy the co-localization at the nuclear envelope was verified for almost the whole nucleus (data not shown). To prove that this co-localization is not due to overexpression of the proteins the inventors used human umbilical artery smooth muscle cells (HUASMCs) and could demonstrate that endogenous NR4A1 also co-localizes at the nuclear envelop with overexpressed ISG12 (FIG. 2b). FIG. 2.a) shows Co-localization of overexpressed NR4A1 with overexpressed ISG12: HUVEC were transfected with EGFP-NR4A1 and mycISG12. After 30 hours cells were fixed, immunostained with a monoclonal anti myc antibody (red fluorescence) and visualized using confocal laser microscopy. In the merged images shown in the figure the co-localized proteins appear as a yellow ring around the nucleus, (n=3). FIG. 2b shows Co-localization of endogenous NR4A1 with overexpressed myc ISG12 in vascular smooth muscle cells (SMCs): 30 hrs after transfection of SMCs with mycISG12, cells were fixed, immunostained with anti NR4A1 (M210, green fluorescence) and anti-myc anti body (red fluorescence) and visualized using confocal laser microscopy. Also endogenous NR4A1 co-localized with overexpressed ISG12 around the nucleus (yellow-orange ring, arrow, (n=2))

Function of ISG12:

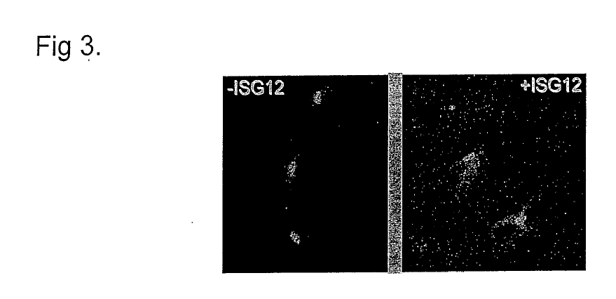

ISG12 decreases nuclear localization of NR4A1: In course of these experiments the inventors observed that in the absence of overexpressed ISG12 NR4A1 was predominantly found in the nucleus, while in the presence of overexpressed ISG12 it was also found in the cytoplasm (FIG. 3). FIG. 3 shows the distribution of overexpressed EGFP-NR4A1 in HUVECs alone and in the presence of overexpressed myc-ISG12 visualized by confocal laser microscopy: NR4A1 (green fluorescence) is predominantly localized in the nucleus in the absence of ISG12 (left panel), while in the presence of overexpressed ISG12, more NR4A1 is seen in the cytoplasma (right panel) (n=2).

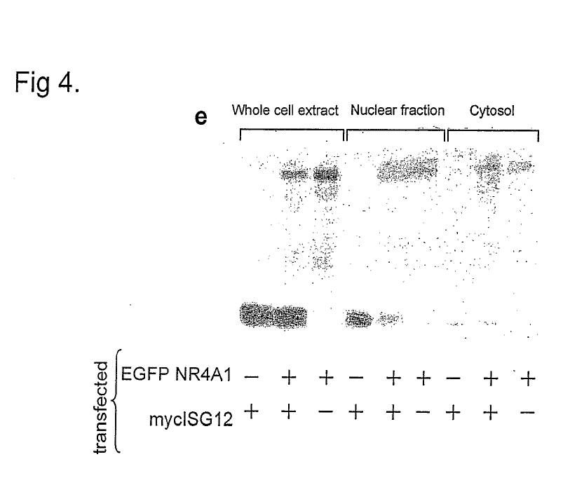

In order to quantify the effect of ISG12 on the cellular localization of NR4A1, the inventors analyzed the distribution of NR4A1 between the cytosol and the nucleus by separating sub-cellular fractions of 293 cells in which EGFP-tagged NR4A1 was overexpressed in the absence or presence of myc-tagged ISG12. In the presence of ISG12 20.8±7.4% (average±SD from 4 experiments; p<0.05) more NR4A1 was found in the cytosol as in the absence of ISG12. FIG. 4 shows differences between cytoplasmic and nuclear concentrations of NR4A1, when overexpressed alone or together with mycISG12: Relative concentrations were determined from Western blots of cell fractionation experiments in 293 cells. A representative example is given (n=3).

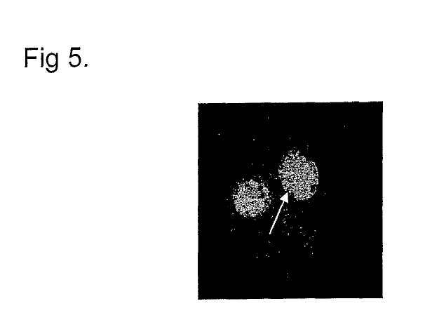

When the same experiments were performed with an EGFP-tagged NR4A1 construct (AA248-580) that cannot be exported from the nucleus²⁴ no influence of ISG12 on subcellular distribution of that construct was seen (FIG. 5). Similarly, the effect of ISG12 on full length NR4A1 was impaired in the presence of leptomycin B that inhibits nuclear export (data not shown). This indicates that ISG12 is a novel interaction partner of NR4A1 localized at the nuclear envelope that decreases nuclear localization of the orphan nuclear receptor NR4A1 by mediating nuclear export. FIG. 5 shows that overexpression of nuclear export deficient EGFP-dnNR4A1 (248-580) together with mycISG12: Co-localization can be observed in HUVECs (yellow ring) around the nucleus but no change in the distribution of the export deficient dnNR4A1 is seen (n=2).

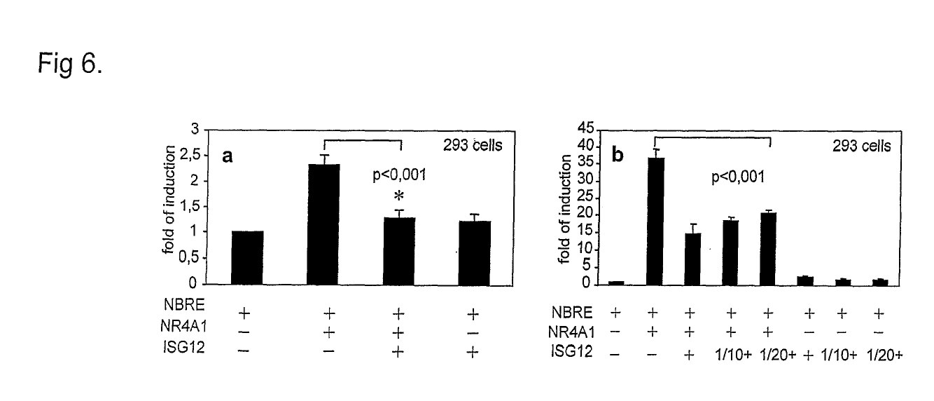

ISG12 decreases transcriptional activity of nuclear receptors: From these data the inventors predicted that ISG12 would also influence transcriptional activity of NR4A1. In order to prove this the inventors employed a luciferase reporter system consisting of a quadruplicated NBRE (NR4A1 monomeric binding site) luciferase construct and analyzed transcriptional activities of NR4A1 in the presence and absence of co-transfected ISG12 (FIG. 6a). Overexpression of NR4A1 more than doubled the reporter gene activity, while in the presence of overexpressed ISG12 NR4A1-induced upregulation of transcriptional activity was significantly reduced. This effect was dependent on the amount of ISG12 transfected as shown by the dose dependency of ISG12 inhibition in FIG. 6b. FIG. 6 shows that ISG12 downregulates NR4A1 transcriptional activities: In a set of experiments the effect of SG12 overexpression on NR4A1 regulated transcription was evaluated; luciferase data were normalized to the expression of renilla and are presented as means±SEM and analyzed by ANOVA. a) A significant (p=0,000298) downregulation of NR4A1 reporter activity by ISG12 is seen in 293 cells (n=4). b) The downregulation of NR4A1 reporter activity by ISG12 was dose dependent as revealed by the decreasing effect of decreasing amounts of transfected ISG12 vector reporter assay in 293 cells, downregulation of NR4A1 was titrated up with decreasing amounts of ISG12 vectors (p=0,000254, (n=2)).

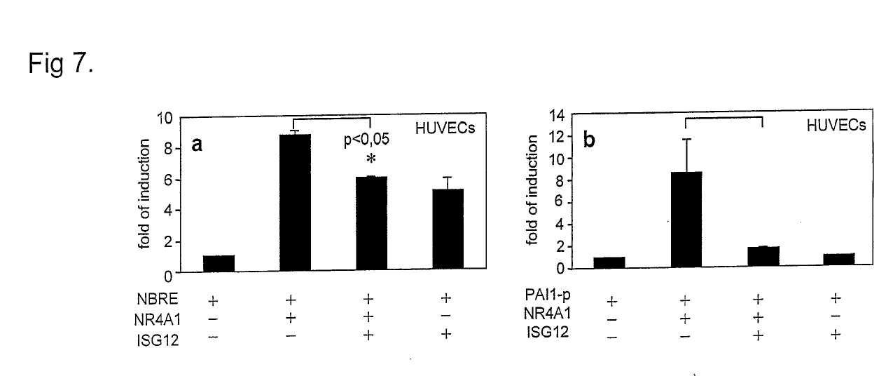

In order to demonstrate that the effect of ISG12 on transcriptional activity of NR4A1 is not restricted to 293 cells and the artificial NBRE construct, the inventors replaced 293 cells by HUVECs and found that the increase in reporter activity induced by NR4A1 was again significantly reduced by ISG12 (FIG. 7a); the inventors also preformed analogous experiments using HUVECs and part of the PAI-1 promoter linked to luciferase, a construct that was used before to demonstrate NR4A1 dependent PAI-1 regulation⁹. As shown in FIG. 7b the PAI-1 reporter activity was upregulated by NR4A1 more than eight fold in the absence of ISG12, while when ISG12 was co-transfected, again the transcriptional activity of NR4A1 was almost abolished. From these data the inventors conclude that ISG12 not only physically interacts with NR4A1 and alters its predominant nuclear localization but it also significantly diminishes NR4A1 transcriptional activities. FIG. 7.a shows Reporter assays preformed in HUVECs also revealed that NR4A1 reporter activity was downregulated by ISG12 (p=0,0118, (n=2)). b) Transcriptional activity of NR4A1 as analyzed using PAI-1 promoter-805-+20 pUB PAI-1 luciferase construct was also downregulated by ISG12 in HUVECs (n=3).

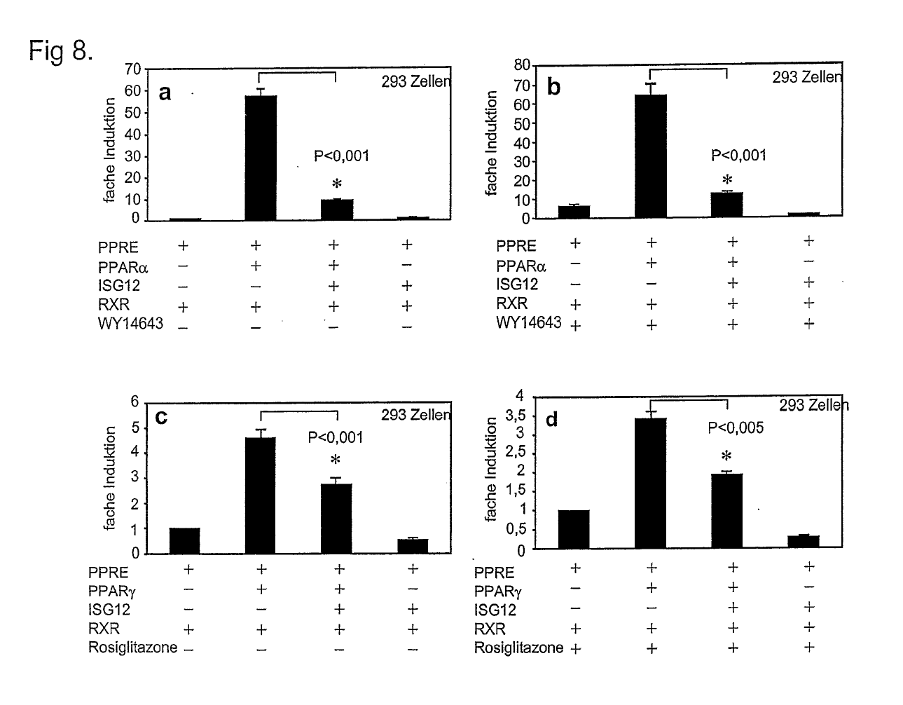

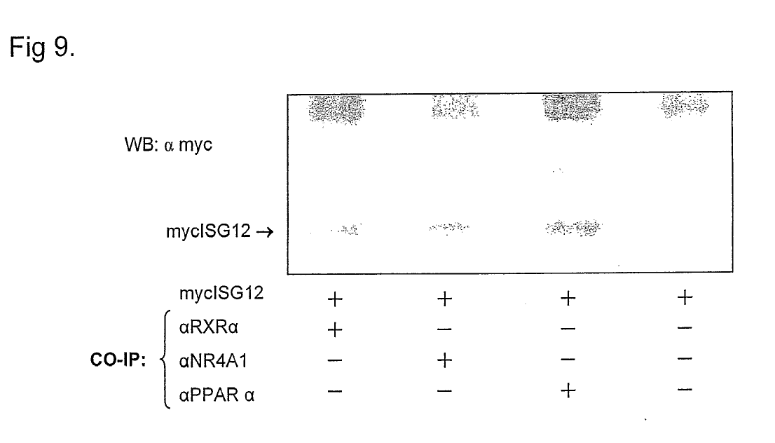

To test the hypothesis that ISG12 not only interferes with the transcriptional activity of NR4A1 but also with that of other nuclear receptors, the inventors again employed a luciferase reporter system consisting of a triplicated PPRE (PPAR response element) luciferase construct and analyzed transcriptional activities of PPARa and PPARg, respectively in the presence and absence of co-transfected ISG12 (FIG. 8). Overexpression of PPARa together with RXR more than 50 fold increased the reporter gene activity, while in the presence of overexpressed ISG12 PPARa-induced upregulation of transcriptional activity was significantly reduced to ˜10 fold (FIG. 8a). Similar effects were seen when PPARa activity was analyzed in the presence of the specific PPARa ligand WY14643 (FIG. 8b). When PPARa was replaced by PPARg again an increase in reporter gene activity was not observed in the presence of overexpressed ISG12 (FIG. 8c) regardless whether the PPARg ligand rosiglitazone was present (FIG. 8d) or not. FIG. 8 shows that ISG12 downregulates transcriptional activity of PPARa and PPARg transcriptional activity and interacts with PPARa and RXRa. a) Transcriptional activity of PPARa as measured in a reporter assay in 293 cells transfected with a PPRE reporter construct and RXRa is significantly downregulated by co-transfection with ISG12 (means±SEM, normalized to renilla expression, p=0,000004, (n=3)). b) Experiments as described in a) were performed in the presence of the PPARa agonist WY-14 643 (100 μM). Also in this case ISG12 downregulates PPARa induced reporter activity (p=0,0000054, (n=3)). c) Experiments performed as in a) using PPARg instead of PPARa show also that co-transfection with ISG12 significantly (p=0,0000088) reduces reporter activity (n=3). d) Downtregulation of PPARg reporter activity by co-transfected ISG12 is also seen in the presence of the PPARg ligand Rosiglitazone (p=0,0023) in analogous experiments(n=2).These data indicated to us that ISG12 not only interacts and downregulates transcriptional activity of NR4A1, but interacts also with other nuclear receptors, downregulating their transcriptional activity. In fact a direct interaction of ISG12 with other nuclear receptors could be found in co-precipitation experiments using antibodies against RXR or PPAR that precipitated myc-tagged ISG12 overexpressed in 293 cells (FIG. 9). FIG. 9 shows co-immunoprecipitation of mycISG12 overexpressed in 293 cells by antibodies against RXRα, NR4A1 and PPARα. Lysates from 293 cells were immunoprecipitated for 4 hrs with the respective rabbit antibodies and a preimmune IgG; Western blotting was performed with a monoclonal mouse anti-myc antibody. With all three specific antibodies ISG12 can be precipitated, while no specific band is seen using the pre-immune IgG (n=2).

Regulated expression of ISG12:

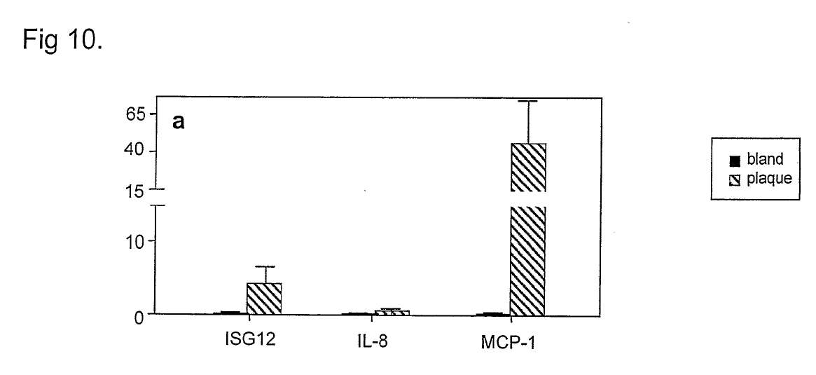

In order to reveal a potential biological role for the interaction of ISG12 with NR4A1 and its inhibitory effect on NR4A1 transcriptional activity, the inventors determined the expression of ISG12 in the vasculature. When specimen obtained from human atherosclerotic vascular lesions defined macroscopically and by the increased expression of Il-8 and MCP-1 (FIG. 10) were analyzed for the expression of ISG12 and compared to non affected adjacent arteries the inventors found an approximately four fold upregulation of ISG12 in the affected areas. From these data the inventors conclude that, ISG12 is upregulated in vascular lesions. FIG. 10 shows regulated expression of ISG12 as analyzed by QT-PCR; relative expression was normalized to levels of PBGD and data are presented as means±SEM. ISG12 is upregulated in human vascular lesions in parallel with the inflammatory marker genes IL-8, MCP-1, (n=2).

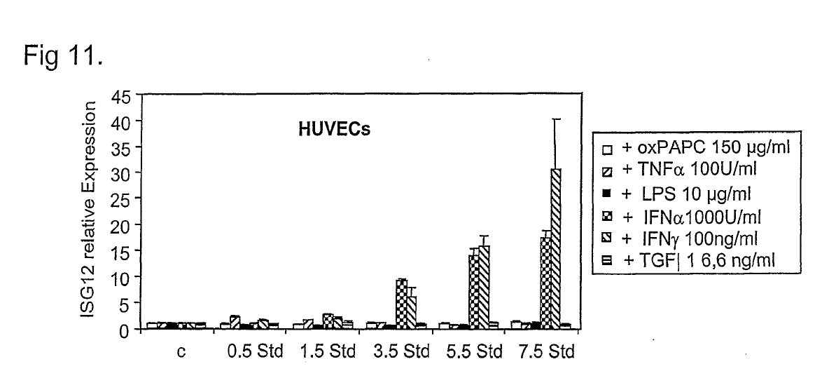

These data further suggest that a cytokine or a mixture of cytokines is present in the arterial lesions that upregulate ISG12. The inventors therefore determined the effect of candidate inflammatory cytokines on the regulation of ISG12 in HWECs in culture (FIG. 11). The inventors found that among the cytokines analyzed only interferon gamma (IFNg) and interferon alpha (IFNa) upregulate ISG12 in endothelial cells more than 10 fold from low levels at resting unstimulated conditions. FIG. 11 shows that expression of ISG12 induced in HUVECs by stimulation with different inflammatory stimuli (ox PAPC 150 μg/ml, TNFa 100 U/ml, LPS 10 μg/ml, IFNa 1000 U/ml, IFNγ 100 ng/ml, TGFβ1 6,6 ng/ml) for 0.5 to 7.5 hours (n=2).

Generation of ISG12 Deficient Mice:

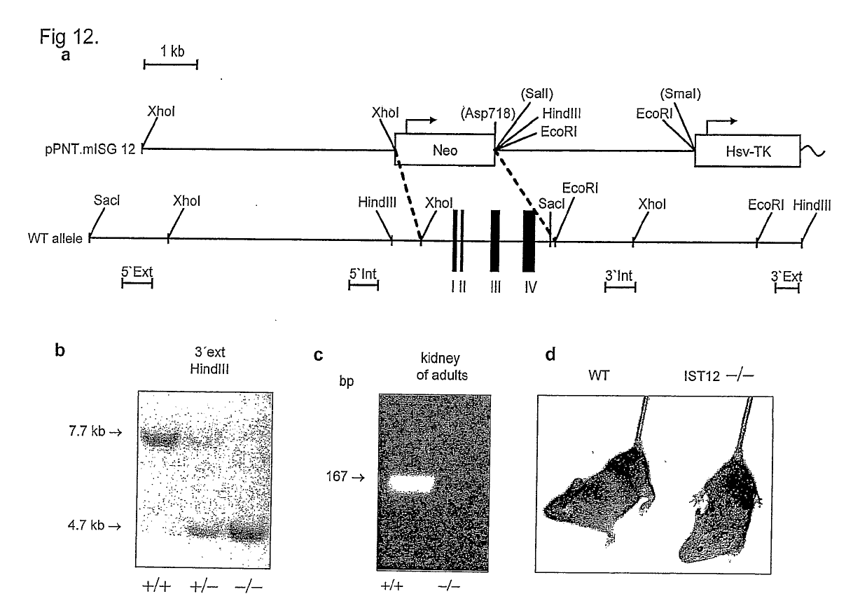

The ISG12^{-/-} mice were generated according to procedures used by us previously²⁶ and outlined in FIG. 12. Obtained ISG12^{+/-} mice and established male and female ISG12^{-/-} mice exhibited no obvious gross phenotypic pathologies (FIG. 12), having normal growth rates, survival and fertility. Routine histological analysis did not reveal any signs of tissue damage. FIG. 12 shows the strategy of ISG12 gene deletion. (a) Black boxes in the genomic structure represent exon sequences. Upon homologous recombination, the neo gene replaces a 2.5 kb genomic fragment encompassing exons I to IV, leading to complete deletion of the ISG12 gene. b), Southern blot analysis of mouse genomic DNA digested with HindIII and hybridized to a 3′-flanking external probe. d) ISG12 mice and wild type.

Use of ISG12 Deficient Mice:

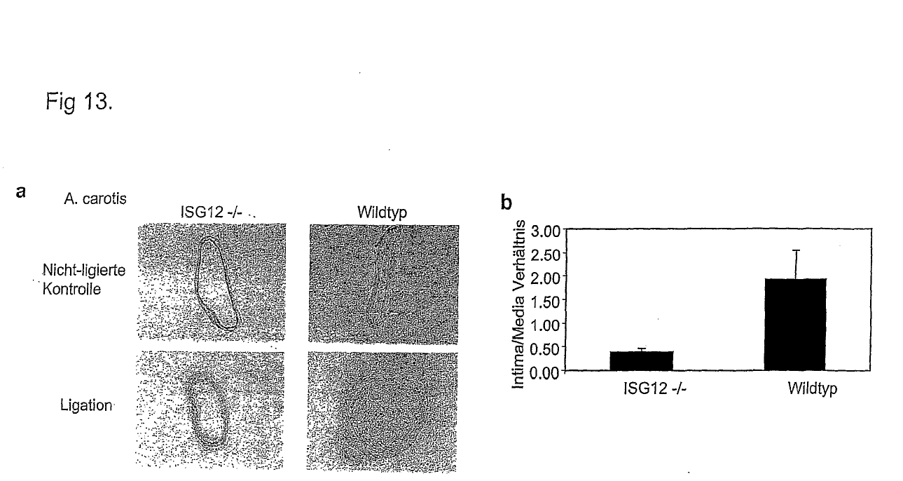

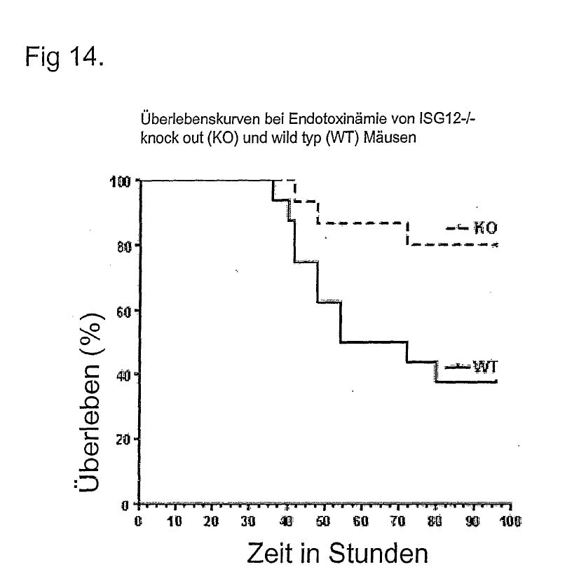

The inventors then used the carotid artery ligation model to determine the effect of ISG12 gene inactivation on neointima formation. As shown in FIGS. 13a and b, ISG12^{-/-} mice exhibited only negligible neointima formation as compared to wild type mice, while in not ligated control carotid arteries no morphological differences were found between wild type and ISG12^{-/-} mice. This indicates that the absence of ISG12 does not influence vascular morphology under basic conditions where ISG12 is expressed only at a low rate also in wild type animals. When injury (ligation) induced upregulation of ISG12 is, however, absent as in the ISG12^{-/-} animals, the lack of ISG12 seems to be beneficial for the vascular repair process. FIG. 13 shows that no neointima formation after carotid artery ligation model was observed in ISG12 deficient mice a) Immunocytochemical image of carotid wall from wt and ISG−/− mice, ligated and unligated, stained with hematoxylin and eosin b) quantification of morphometry analysis, differences between neointima to media ratios in wt and ISG12^{-/-} were statistically significant, *p=0,0038. An other example for the effect of ISG12 deficiency is given in FIG. 14. Here the survival of wild type mice upon intraperitoneal injection of endotoxin is compared to ISG12^{-/-} mice. ISG12 deficient mice show as significant better survival as wild type mice.

Single Nucleotide Polymorphism in ISG12 Correlate with Vascular Disease

In a human genetic study, the inventors analyzed three single nucleotide polymorphisms (SNP) in the human ISG12 gene on chromosome 4q (official gene name: interferon, alpha-inducible protein 27 (IFI27), NM_{—}005532). Of these, two were polymorphic in our sample (rs2239644 with 15.0% heterozygosity and rs2799 with 4.1% heterozygosity). These polymorphisms were analyzed for association with coronary heart disease (past heart attack or angina), stroke, diabetes type II, arterial hypertension and hypercholesterinemia in 853 Caucasian subjects recruited at the Max-Planck Institute of Psychiatry in Munich, Germany within a case-control study for unipolar recurrent depression

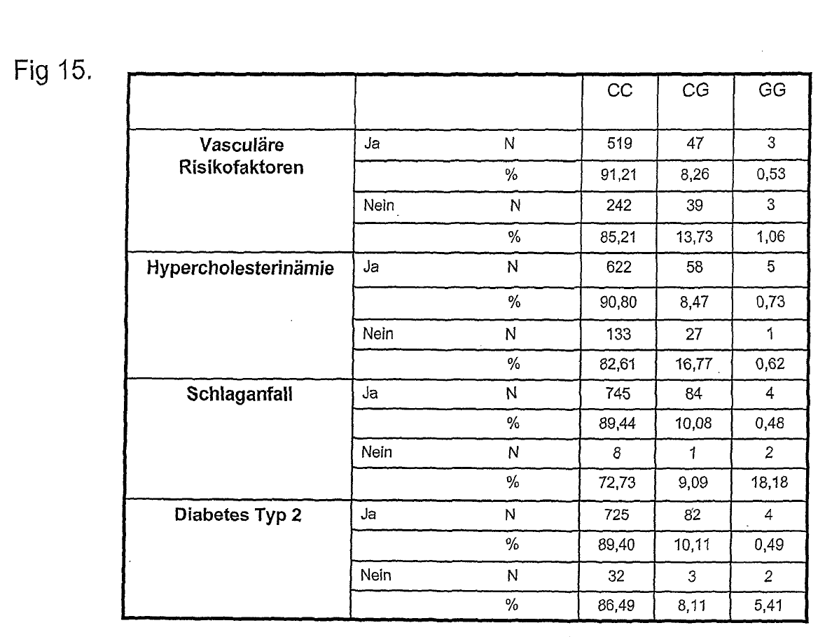

The inventors found a significant association of rs2799, located in the 3′UTR of the gene, with the presence of hypercholesterolemia (p=0.006), diabetes type 2 (p=0.00058) and stroke (p=0.0008) but not high blood pressure or the presence of heart attacks or angina. All associations remained significant in a logistic regression analysis when controlling for age, sex and depression status. This suggested to us that the ISG12 gene might also be important for human vascular diseases. FIG. 15 shows the distribution of rs2799 genotypes and presence of vascular risk factors.

Discussion

The inventors found a novel modulator of transcriptional activities of a set of nuclear receptors, the interferon regulated gene 12 (ISG12) that interacts with NR4A1, is strongly upregulated by INFa and INFg and found over-expressed in vascular lesions. The inventors furthermore show here that ISG12, a proposed nuclear envelope protein with hitherto unknown function²³, influences the nucleo-cytoplasmic distribution of NR4A1 and represses its transcriptional activity. When mice made deficient in ISG12 were analyzed for their susceptibility to develop vascular lesions the inventors found that ISG12^{-/-} mice were resistant to restenosis after carotid artery ligation. When mice double deficient in ISG12 and NR4A1, however, were used, these mice again exhibited restenosis upon carotid artery ligation to a similar extent as did wild type mice. This indicates that a major target for ISG12 in vascular pathologies is NR4A1. Further support for a possible importance of ISG12 comes from human genetics studies. The inventors could show a strong association between the intronic ISG12 single nucleotide polymorphism (SNP), rs2799 and the presence of hypercholesterolemia, diabetes type 2 and stroke. In fact the GG genotype of this polymorphism appeared to be protective against these disorders. This pointed us to analyze the effect of ISG12 on the adopted nuclear receptors PPARa and PPARg. ISG12 downregulated transcriptional activities of PPARa and PPARg in a similar way as it did with the transcriptional activity of NR4A1. Similarly ISG12 interacted also physically with these nuclear receptors. These data indicate that ISG12 is a novel gene upregulated in the vasculature upon injury that contributes to vascular pathologies by increasing nuclear export of “beneficial” nuclear receptors thereby down-regulating their transcriptional activities. ISG12 therefore might represent a target for novel therapeutic strategies for the treatment of vascular diseases.

Methods:

Cell Culture, Vectors, Transfections and Reagents.

Human umbilical vein endothelial cells (HUVECs) and human umbilical artery smooth muscle cells (HUASMCs) were cultured as previously described and used until the 5^{th} passage. HUVEC were transfected using Lipofectamine Plus® reagent (Invitrogen, Carlsbad, Calif.) according to the manufacturer's protocol using 1.5 μg DNA. For stimulation cells were starved over night with M199 containing 3% serum prior to the experiments. Human embryonic kidney—293 cells were obtained and cultured as recommended (ATCC, Manassas, Va.). 293 cells were transiently transfected for luciferase reporter assays or for cell fractionation experiments using calcium-phosphate³¹ and 3 μg DNA.

Human ISG-12 full length wild type, amino acids 1-122, was cloned into pCMV Myc (BD Biosciences-Clontech, Paolo Alto, Calif.) and NR4A1 full length (1-598) and dominant negative NR4A1 (248-580) was cloned into EGFP-C1 (BD Biosciences-Clontech, Paolo Alto, Calif.). The luciferase reporter construct NBRE 4× (pGL3 plasmid containing four canonical NBRE sites) and pc DNA 3.1 NR4A1 (1-598 amino acids, full length, wt) were described previously⁹. Mammalian expression vectors for human retinoid X receptor alpha (RXRα), mouse PPARg (mPPARg, mouse PPARa (m PPARa), and the luciferase reporter construct PPRE₃-TK-LUC³² were obtained from L. Nagy, Debrecen, Hungary.

Yeast Library Construction and Yeast 2 Hybrid Screening

The construction of a yeast library was performed using the MATCHMAKER Library Construction and Screening Kit (BD Biosciences Clontech, Paolo Alto, Calif.) according to the manufacturer's recommendations. Briefly, total RNA was isolated from activated human uterus microvascular endothelial cells (HUMEC) stimulated with endotoxin for 4, 9 and 16 hours and tumor necrosis factor alpha (TNFa) for 2, 4, 9, and 16 hours, respectively with Trizol (Invitrogen, Carlsbad, Calif.); mRNA was purified with oligo (dT) labeled magnetic beads (Dynal, Oslo, Norway). Upregulation of Il-8 as revealed by quantitative rtPCR (Roche Diagnostic, Basel, Switzerland) confirmed efficient stimulation after TNFa or LPS treatment at the respective time points (data not shown). mRNAs isolated from the differently stimulated cells were pooled and first strand synthesis was performed by the SMART technology based on template switching using modified oligo (dT) primers in combination with MMLV RT. Following PCR, cDNAs and linearized pGADT7-Rec vector containing the GAL4 activation domain (AD) were co-transformed into AH109 yeast strain (MATa, trp1-901, leu2-3, 112, ura3-52, his3-200, gal4D, gal80D, LYS2::GAL1UAS-GAL1TATA-HIS3, GAL2UAS-GAL2TATA-ADE2, URA3::MEL1UAS-MEL1TATA-lacZMEL1).

For screening, a construct from NR4A1 containing 248-557 amino acid lacking the transactivation domain 2 was cloned into pGBKT7 containing the GAL4 binding domain (BD, Paolo Alto, Calif.)), and the resulting construct was confirmed by sequencing with ABI Prism® Big Dye™ Terminator Cycle Sequencing Ready Reaction Kit (Applied Biosystems, Foster City, Calif.) on 310 Genetic Analyzer (Perkin Elmer, Wellesley, Mass.). Autoactivation of the bait was excluded by co-transformation with empty AD containing the vector plated on selective agar. The bait construct was transformed into Y187 yeast strain (MATa, ura3-52, his3-200, ade2-101, trp1-901, leu2-3, 112, gal4D, gal80D, met-, URA3::GAL1UAS-GAL1TATA-lacZMEL1). Mating was performed according to the manufacturers protocol with a AH109 yeast strain from the microvascular endothelial cell library containing 2×10⁶ independent clones (Clontech, Paolo Alto, Calif.) with bacterial RNA as carrier nucleotides as described by Brondyk and Macara³³ resulting in about 1,6×10⁶transformants. Positive colony selection was carried out on SD medium lacking leucine, tryptophane, adenine and histidine, and subsequently in the presence of 35 mM 3-amino-1,2,4-triazole (3-AT). Plasmid DNA from the yeast was prepared by the method of Liang and Richardson³⁴ followed by electrotransformation into HB101 bacteria. Plasmids were isolated from bacteria by Fast plasmid™ mini kit (Eppendorf, Hamburg, Germany) and sequenced by ABI Prism® Big Dye™ Terminator Cycle Sequencing Ready Reaction Kit (Applied Biosystems, Foster City, Calif.) on 310 Genetic Analyzer (Perkin Elmer, Wellesley, Mass.). Positive interactions of bait and prey were confirmed by retransformation in yeast.

Immunoprecipitation, Western-Blotting

Transfected 293 cells were washed with phosphate-buffered saline and lysed for 30 min with a cell lysis buffer containing 2.7 mM KCl, 1.5 mM KH₂PO₄, 9.2 mM Na₂HPO₄.2H₂O, 150 mM NaCl, 0.7% NP40, 0.3% Triton X-100 and complete protease inhibitor cocktail (Roche Diagnostic, Basel, Switzerland). Concentration of NaCl was then adjusted to 350 mM and cell lysates were then incubated for 3 h at +4° with 2 μg of appropriate antibody: anti NR4A1 M210 (Santa Cruz Biotechnology, Santa Cruz, Calif.), anti PPARα H-98 (Santa Cruz Biotechnology, Santa Cruz, Calif.), antRXRα ΔN 197 (Santa Cruz Biotechnology, Santa Cruz, Calif.) or rabbit immunoglobulin fraction (DAKO, Milan, Italy) and protein A-Sepharose beads (Amersham Biosciences, Buckinghamshire, UK). After five times washing with 1×PBS, beads were then re-suspended in Laemmli buffer. Collected samples were subjected to 12% SDS-polyacrylamide gel electrophoresis and transferred to a polyvinylidene difluoride membrane (Millipore, Bedford, Mass.). Detection of immunoreactive bands with anti myc antibodies for co-immunoprecipitation (Oncogene, San Diego, Calif.) was done with ECL Plus (Amersham Biosciences, Buckinghamshire, UK) according to the manufacturer's protocol.

Immunofluorescence and Confocal Microscopy

HUVECs or SMCs were grown to 50-80% confluency in chambers slides (Nalge Nunc International, Naperville, Ill.) and transfected with EGFP-NR4A1 (1-598) or EGFP-dominant negative NR4A1 (248-580) and mycISG12 alone or together in combination Thirty hours after transfection cells were fixed in 4% paraformaldehyde, afterwards permeabilized with Triton X-100 an then stained for mycISG12 with anti-myc antibody (Oncogene,San Diego, Calif.) at a dilution of 1:100 and Alexa flour 568 conjugated goat anti-mouse second antibody in 1:200 dilution (Molecular Probes, Eugene, Oreg.). Presence of enhanced green fluorescent protein (EGFP) from the co-transfected vector pEGFP-NR4A1 full length and pEGFP-dnNR4A1 was determined on a Olympus AX-70 microscope (HUASMCs) or LSM510 (HUVECs) microscope (Zeiss, Germany) at 488 nm excitation and 512 nm emission wave length and separated from the alexa flour 568 label (excitation wavelength 543 nm, emission wavelength 603 nm).

Cell Fractionation

Human embryonic kidney 293 cells were grown in 6-wells to sub-confluence and then transfected with EGFP-NR4A1 and mycISG12 together or separately. Cell fractionation was performed 30 hours after transfection, using MS buffer (210 mM mannitol, 70 mM sucrose, 5 mM Tris-HCl, pH 7.5 and 1 mM EDTA) containing 1% protease inhibitor cocktail. After homogenizing the cells with a 27 gauge needle 10 times, samples were spun at 1300×g for 10 minutes at 4° C. to pellet the nuclei. Supernatants representing cytoplasmic and heavy membrane fractions was decanted and centrifuged once more for 30 min at 16000×g to separate the cytoplasm; afterwards membranes were pelleted. Nuclear pellets were washed once more with ice-cold PBS and re-suspended in MS buffer. The nuclei were purified by a second centrifugation at 1300×g an then the nuclear pellet was lysed by a buffer containing: 2.7 mM KCl, 1.5 mM KH₂PO₄, 9.2 mM Na₂HPO₄.2H₂O, 150 mM NaCl, 0.7% NP40, 0.3% Triton X-100 and complete protease inhibitor cocktail (Roche Diagnostic, Basel, Switzerland). Separated nuclear and cytoplasmic fractions were then diluted in Laemmli buffer and prepared for Western blotting after separation on a 9% SDS PAGE using an anti EGFP antibody (Abcam, Cambridge, UK).

Luciferase Assays

Human embryonic kidney 293 cells or HUVECs were transfected as above with renilla vector as an internal control. In case of experiments where PPARs were stimulated by their ligand, media was exchanged 9 hours after transfection and stimulation with WY-14 643 (Biomol, Hamburg, Germany) or rosiglitazone maleate (Alexis biochemicals, Lausen, Switzerland) was performed 21 hrs before luciferase and renilla assays were performed. Luciferase and renilla assays were performed 30 hrs after transfection of the cells on cell lysates with a Dual Luciferase Assay Kit (Promega, Madison, Wis.) according to the manufacturer's instructions. Luciferase activity was normalized to the respective renilla values. All experiments were done in triplicate wells and at least twice.

RNA isolation and relative quantitative reverse transcriptase-polymerase chain reaction (Q-PCR)

RNA from stimulated HUVECs and from human and mouse tissue samples was extracted using Trizol (Invitrogen, Carlsbad, Calif.) reagent. Total RNA (900 ng) was reverse transcribed with MuLV-reverse transcriptase using the Gene Amp RNA PCR kit (Applied Biosystems, Foster City, Calif.) and oligo dT₁₆ primers. Isolated arterial tissue from mice and was immersed into ice-cold RNAlater (Ambion, Austin Tex.) and stored in buffer at −70° C. until isolation. RNA isolation from pooled arterial tissue (n=3) was done as described previously³⁵ and 350 ng RNA were reverse transcribed with the same kit as above.

The mRNA sequences for the genes to be analyzed were obtained from GenBank. The primers, including mouse and human, were designed using the PRIMER3 software (Whitehead Institute for Biomedical Research, Cambridge, Mass.) The following forward (F) and reverse (R) primers were used for human ISG12: F, 5′-TGTGATTGGAGGAGTTGTGG -3′; R, 5′-GAACTTGGTCAATCCGGAGA -3′; mouse ISG125′-CTG CCA TAG GAG GAG CTC TG-3′ and 5′-ATG GCA TTT GTT GAT GTG GA-3′; human MCP-1³⁶; mouse MCP-1³⁷; human IL-8 F, 5′-CTC TTG GCA GCC TTC CTG ATT-3′; R, 5′-TAT GCA CTG ACA TCT AAG TTC TTT AGC A-3′. Q-PCR was performed by LightCycler technology using the Fast Start SYBR Green I kit for amplification and detection (Roche Diagnostics, Basel, Switzerland). In all assays, cDNA was amplified using a standardized program (10′ denaturing step and 55 cycles of 5′ at 95° C.; 15′ at 65° C., and 15′ at 72° C.; melting point analysis in 0.1° C. steps; final cooling step). Each LightCycler capillary was loaded with 1.5 μL DNA Master Mix; 1.8 μL MgCl2 (25 mM); 10.1 μL H2O; and 0.4 μL of each primer (10 μM). The final amount of cDNA per reaction corresponded to 2.5 ng total RNA used for reverse transcription. Relative quantification of target gene expression was performed using a mathematical model by Pfaffl³⁸. The expression of the target molecule was normalized to the expression of PBGD with primers for human PBGD F, 5′-TCG AGT TCA GTG CCA TCA TC-3′ and PBGD R, 5′-CAG GTA CAG TTG CCC ATC CT-3′ or mouse PBGD³⁹.

Generation of ISG12^{-/-} and Double Deficient ISG12^{-/-} NR4A1^{-/-} Mice

The ISG12^{-/-} mice were generated according to procedures used by us previously²⁶. The murine gene consists of four exons, separated by three relatively small introns (introns I to III), as outlined in FIG. S1a. The cDNA sequence of mISG12 was used to design primers specific for different regions of mISG12 gene. Using 129S/v genomic DNA as a template, the PCR reactions were optimized and used for screening of the genomic library generated by HindIII partial digestion of a 129S/v mouse genomic DNA library and cloned into RPCI.22 BAC vector (Invitrogen, Carlsbad, Calif.). Obtained genomic clones were used to prepare targeting vectors to inactivate the ISG12 gene in embryonic stem (ES) cells. In total, an 8.5 kb homologous sequence of the ISG12 gene was introduced into a parental pPNT vector containing a neomycin phosphotransferase (neo) cassette and a herpes simplex virus pPNT.mISG12. Briefly, in the first step, a 4.7 kb XhoI fragment containing 5′ UTR of mISG12 gene was ligated into the XhoI site of pPNT generating plasmid pPNT1.mISG12. Subsequently, a 3.8 kb EcoRI fragment encompassing 3′ UTR of mISG12 gene was cloned into the EcoRI site of pBluescript®II KS(+/−) vector (Stratagene, La Jolla, Calif.). Subsequently, a 3.85 kb SalI-SmaI fragment from this vector was Klenow filled and ligated into Asp718 site/Klenow filled pPNT1.mISG12, yielding a targeting vector pPNT.mISG12. Transfection of targeting vectors resulted in four out of 200 G418/Ganciclovir-double-resistant clones that underwent the desired homologous recombination, as confirmed by comprehensive Southern blotting of the isolated genomic DNA from R1 embryonic stem (ES) cells derived from the 129S/v mouse strain (received from A. Nagy, Samuel Lunenfeld Institute, Toronto, Canada). Chimeric mice (F0), obtained by 8 cells stage embryo aggregation of the targeted ES cell clones, were test-bred for germ line transmission with Swiss mice. They transmitted the deleted ISG12 allele to their offspring (50% 129S/v:50% Swiss genetic background), yielding ISG12^{+/-} mice. Intercrossing of these mice resulted in ISG12^{-/-} mice, as identified by Southern blot analysis of tail tip DNA using the 3′-external probe (FIG. S1b). Correct inactivation of the ISG12 gene was further confirmed with additional digests using 5′-external, 5′-internal, neo-specific and 3′-flanking internal probes (not shown) and by RT-PCR (FIG. S1c). Wild-type and heterozygous control mice used in the experiments were littermates of the corresponding knockouts. All experiments were performed in compliance with institutional guidelines and approved by the respective university committee (Austrian animal experimental law permission No. 169/1999).

To generate ISG12^{-/-} NR4A1^{-/-} double deficient mice and respective control mice with single ISG12^{-/-} or NR4A1^{-/-} deficiencies, ISG12^{-/-} mice were bred with Nr4A1^{-/-} mice (in B6 background, kindly provided by J. Milbrandt from the Department of Pathology, Washington University School of Medicine, St. Louis, USA). Double heterozygous mice were obtained and subsequently bred to obtain ISG12^{+/+}NR4A1^{+/+}, ISG12^{+/+}NR4A1^{-/-}, ISG12^{-/-} NR4A1^{+/+} and ISG12^{-/-} NR4A1^{-/-} mice. These mice were used for analyses. Genotyping of mice with respect to the NR4A1allele was done by Southern blotting using an exon 2 specific probe and BamHI genomic digests yielded a 4.9 kb and a 6.6 kb fragment for the wild-type and recombined allele, respectively.

Animal Experiments

For the restenosis mouse model, mice at 8 weeks of age were subjected to carotid artery ligation according to the published procedure by Kumar and Lindner⁴¹. Briefly, the left common carotid artery of ketamine/xylazine anesthetized mice was ligated near the distal bifurcation (n=4 to 7). At 2.5 or 4 weeks after ligation, mice were anesthetized and subsequently perfused via the heart with PBS and carotid arteries were harvested.

For the atherosclerotic lesion model in mice, Apo E^{-/-} and wt mice at the age of 6 months were anesthetized with ketamine/xylazine and than perfused via the heart with PBS and aortic arches and heart valves were collected.

Morphometry

The ligated artery was sectioned from the ligature to the aortic arch, fixed with 4% paraformaldehyde and than stained with haematoxylin and eosin or stained for smooth muscle actin (clone 1A4-FITC, Sigma) and counterstained with DAPI (4,6 diamidino-2-phenylindole; Vector Laboratories, Burlingame, Calif.). A standardized reference point was set at which the vessel structure was not distorted by the ligature and the elastic laminae were intact. Cross sections 0.7 mm, 1.7 mm and 2.7 mm from the reference point were morphometrically analyzed using the AnalySiS® software package (Soft Imaging System; Muinster, Germany) on digital images of the vessel (green channel: smooth muscle actin, blue channel: DAPI and red channel: autofluorescence of the lamina elastica), obtained with a F-View camera on an Olympus AX-70 microscope. The circumferences of the lumen, internal elastic lamina and external elastic lamina were measured, and media area, neointimal area, and neointima/media ratio were calculated

Patient Recruitment

853 individuals were recruited at the Max-Planck Institute of Psychiatry in Munich, Germany within a case-control study for unipolar recurrent depression. All individuals were Caucasian and 88% of German origin. In patients, psychiatric diagnosis was ascertained by WHO-certified raters according to DSM-IV using the Schedule for Clinical Assessment in Neuropsychiatry (SCAN). Matched controls were selected randomly from a Munich-based community sample and screened for the presence of anxiety and affective disorders using the Composite International Diagnostic Screener. In addition, the presence of 35 different medical, neurological and psychiatric disorders in all depressed patients and healthy controls and their first grade relatives was assessed using a self-report sheet. For this study the presence of a heart attack or angina, a stroke, high blood pressure and high blood cholesterol and diabetes type 2 in the patients and controls was recorded and used for statistical analysis. Of the 853 individuals, 366 presented with unipolar depression, 35 reported a history of coronary heart disease or angina, 161 hypercholesterinemia, 187 high blood pressure, 37 diabetes type 2 and 11 a history of stroke. 67.3% were female and the mean age was 50.8 years (SD=13.8).

Genotyping

On enrollment in the study, 40 ml of EDTA blood were drawn from each patient and each healthy control. DNA was extracted from fresh blood using the Puregene™ whole blood DNA-extraction kit (Gentra Systems Inc; MN).

Three SNPs (rs223944, rs223945 and rs2799) were selected in the human homologue of ISG12, IFI27 (NM_{—}005532) located on chromosome 14q32 using dbSNP (http://www.ncbi.nlm.nih.gov:80/). The SNP search tool at http://ihg.gsf.de/ihg/snps.html was used to download SNP sequences from public databases. Hardy Weinberg equilibrium (HWE) was tested in the psychiatric control group. Both SNPs were in Hardy Weinberg Equilibrium in this group of individuals (p=0.20 for rs2799 and p=0.44 for rs223944).

Genotyping was performed on a MALDI-TOF mass-spectrometer (MassArray® system) employing the Spectrodesigner software (Sequenom™; CA) for primer selection and multiplexing, and the homogeneous mass-extension (hMe) process for producing primer extension products. Genotyping was performed at the Genetics Research Centre GmbH, Munich, Germany. All primer sequences are available upon request.

Statistical Analysis

Experimental values are expressed as average±SEM. Statistical significance of differences in reporter assays were analyzed by Student's unpaired two-tailed t-test.

The significance of differences in morphometric analysis was determined by using the nonparametric Mann-Whitney 2-tailed U test and expressed as a probability value.

IFI27 SNPs were tested for association with the presence of a heart attack or angina, a stroke, high blood pressure and high blood cholesterol and diabetes type 2. Analyses for these case/control associations were performed using the Armitage's trend test with the statistical tool at (http://ihg.gsf.de/cgi-bin/hw/hwa2.pl). The statistical tests for association in this web-based tool are adapted from Sasieni⁴². In addition, association analyzes were performed calculating a logistic regression using SPSS for Windows (Releases 11, SPSS Inc., Chicago, Ill.) testing effects on the genotypic level with age, sex and depression status as covariates.

GenBank Accession Numbers:
Protein Homo sapiens ISG12, GI:55925614; Mus musculus ISG12 GI:44771124; Homo sapiens Nur77 GI:21361342,
mRNA Homo sapiens ISG12 GI:55925613; Mus musculus ISG12 GI:44771123; Homo sapiens Nur77 GI:21361342 mRNA Homo sapiens Hydroxymethylbilane synthase HMBS (PBGD) GI:66933007 mRNA Homo sapiens Interleukin 8 (IL8) GI:28610153 target for novel therapeutic and diagnostic strategies in inflammation, metabolic and vascular diseases.

The disclosure and content of the following references is included in this application by reference.

### REFERENCE LIST

1. Libby, P. Inflammation in atherosclerosis. Nature 420, 868-874 (2002).
2. Hansson, G. K. Inflammation, atherosclerosis, and coronary artery disease. N. Engl. J. Med. 352, 1685-1695 (2005).
3. Gerrity, R. G. The role of the monocyte in atherogenesis: I. Transition of blood-borne monocytes into foam cells in fatty lesions. Am. J. Pathol. 103, 181-190 (1981).
4. Gerrity, R. G. The role of the monocyte in atherogenesis: II. Migration of foam cells from atherosclerotic lesions. Am. J. Pathol. 103, 191-200 (1981).
5. Libby, P. et al Macrophages and atherosclerotic plaque stability. Curr. Opin. Lipidol. 7, 330-335 (1996).
6. Frostegard, J. et al Cytokine expression in advanced human atherosclerotic plaques: dominance of pro-inflammatory (Th1) and macrophage-stimulating cytokines. Atherosclerosis 145, 33-43 (1999).
7. Libby, P., Sukhova, G., Lee, R. T. & Galis, Z. S. Cytokines regulate vascular functions related to stability of the atherosclerotic plaque. J. Cardiovasc. Pharmacol. 25 Suppl 2, S9-12 (1995).
8. Bourcier, T., Sukhova, G. & Libby, P. The nuclear factor kappa-B signaling pathway participates in dysregulation of vascular smooth muscle cells in vitro and in human atherosclerosis. J. Biol. Chem. 272, 15817-15824 (1997).
9. Gruber, F. et al Direct binding of Nur77/NAK-1 to the plasminogen activator inhibitor 1 (PAI-1) promoter regulates TNF alpha-induced PAI-1 expression. Blood 101, 3042-3048 (2003).
10. de Vries, C. J., van Achterberg, T. A., Horrevoets, A. J., ten Cate, J. W. & Pannekoek, H. Differential display identification of 40 genes with altered expression in activated human smooth muscle cells. Local expression in atherosclerotic lesions of smags, smooth muscle activation-specific genes. J. Biol. Chem. 275, 23939-23947 (2000).
11. Pei, L., Castrillo, A., Chen, M., Hoffmann, A. & Tontonoz, P. Induction of NR4R orphan nuclear receptor expression in macrophages in response to inflammatory stimuli. J. Biol. Chem. (2005).
12. Arkenbout, E. K. et al Protective function of transcription factor TR3 orphan receptor in atherogenesis: decreased lesion formation in carotid artery ligation model in TR3 transgenic mice. Circulation 106, 1530-1535 (2002).
13. Cunard, R. et al WY14,643, a PPAR alpha ligand, has profound effects on immune responses in vivo. J. Immunol. 169, 6806-6812 (2002).
14. Li, A. C. et al Differential inhibition of macrophage foam-cell formation and atherosclerosis in mice by PPARalpha, beta/delta, and gamma. J. Clin. Invest 114, 1564-1576 (2004).
15. Li, A. C. & Glass, C. K. J. Lipid Res. 45, 2161-2173 (2004).
16. Kagaya, S. et al Prostaglandin A(2) Acts as a Transactivator for NOR1 (NR4A3) within the Nuclear Receptor Superfamily. Biol. Pharm. Bull. 28, 1603-1607 (2005).
17. Sohn, Y. C., Kwak, E., Na, Y., Lee, J. W. & Lee, S. K. Silencing mediator of retinoid and thyroid hormone receptors and activating signal cointegrator-2 as transcriptional coregulators of the orphan nuclear receptor Nur77. J. Biol. Chem. 276, 43734-43739 (2001).
18. Wu, W. S., Xu, Z. X., Ran, R., Meng, F. & Chang, K. S. Promyelocytic leukemia protein PML inhibits Nur77-mediated transcription through specific functional interactions. Oncogene 21, 3925-3933 (2002).
19. Berger, J. & Moller, D. E. The mechanisms of action of PPARs. Annu. Rev. Med. 53, 409-435 (2002).
20. Wansa, K. D., Harris, J. M. & Muscat, G. E. The activation function-1 domain of Nur77/NR4A1 mediates transactivation, cell specificity, and coactivator recruitment. J. Biol. Chem. 277, 33001-33011 (2002).
21. Ebner, K., Bandion, A., Binder, B. R., De Martin, R. & Schmid, J. A. GMCSF activates NF-{kappa}B via direct interaction of the GMCSF-receptor with I{kappa}B kinase{beta}. Blood (2003).
22. Hofer-Warbinek, R. et al A highly conserved proapoptotic gene, IKIP, located next to the APAF1 gene locus, is regulated by p. 53. Cell Death Differ 11, 1317-1325 (2004).
23. Martensen, P. M. et al The interferon alpha induced protein ISG12 is localized to the nuclear membrane. Eur. J. Biochem. 268, 5947-5954 (2001).
24. Gruber, F. et al Direct binding of Nur77/NAK-1 to the plasminogen activator inhibitor 1 (PAI-1) promoter regulates TNFalpha-induced PAI-1 expression. Blood 101, 3042-3048 (2003).
25. Breuss, J. M. et al Activation of nuclear factor-kappa B significantly contributes to lumen loss in a rabbit iliac artery balloon angioplasty model. Circulation 105, 633-638 (2002).
26. Uhrin, P. et al Disruption of the protein C inhibitor gene results in impaired spermatogenesis and male infertility. J. Clin. Invest 106, 1531-1539 (2000).
27. Crawford, P. A., Sadovsky, Y., Woodson, K., Lee, S. L. & Milbrandt, J. Adrenocortical function and regulation of the steroid 21-hydroxylase gene in NGFI-B-deficient mice. Mol. Cell Biol. 15, 4331-16 (1995).
28. Lee, S. L., Wang, Y. & Milbrandt, J. Unimpaired macrophage differentiation and activation in mice lacking the zinc finger transplantation factor NGFI-A (EGR1). Mol. Cell Biol. 16, 4566-4572 (1996).
29. Chinetti-Gbaguidi, G., Fruchart, J. C. & Staels, B. Role of the PPAR family of nuclear receptors in the regulation of metabolic and cardiovascular homeostasis: new approaches to therapy. Curr. Opin. Pharmacol. 5, 177-183 (2005).
30. Staels, B. & Fruchart, J. C. Therapeutic roles of peroxisome proliferator-activated receptor agonists. Diabetes 54, 2460-2470 (2005).
31. Perkins, N. D. et al A cooperative interaction between NF-kappa B and Sp1 is required for HIV-1 enhancer activation. EMBO J. 12, 3551-3558 (1993).
32. Kliewer, S. A. et al Differential expression and activation of a family of murine peroxisome proliferator-activated receptors. Proc. Natl. Acad. Sci. U.S.A 91, 7355-7359 (1994).
33. Brondyk, W H. & Macara, I. G. Use of two-hybrid system to identify Rab binding proteins. Methods Enzymol. 257, 200-208 (1995).
34. Liang, Q. & Richardson, T. A simple and rapid method for screening transformant yeast colonies using PCR. Biotechniques 13, 730-2, 735 (1992).
35. Furnkranz, A. et al Oxidized phospholipids trigger atherogenic inflammation in murine arteries. Arterioscler. Thromb. Vasc. Biol. 25, 633-638 (2005).
36. Leonarduzzi, G. et al Oxysterol-induced up-regulation of MCP-1 expression and synthesis in macrophage cells 1. Free Radic. Biol. Med. 39, 1152-1161 (2005).
37. Furnkranz, A. et al Oxidized phospholipids trigger atherogenic inflammation in murine arteries. Arterioscler. Thromb. Vasc. Biol. 25, 633-638 (2005).
38. Pfaffl, M. W. A new mathematical model for relative quantification in real-time RT-PCR. Nucleic Acids Res. 29, e45 (2001).
39. Furnkranz, A. et al Oxidized phospholipids trigger atherogenic inflammation in murine arteries. Arterioscler. Thromb. Vasc. Biol. 25, 633-638 (2005).
40. Labrada, L., Liang, X. H., Zheng, W., Johnston, C. & Levine, B. Age-dependent resistance to lethal alphavirus encephalitis in mice: analysis of gene expression in the central nervous system and identification of a novel interferon-inducible protective gene, mouse ISG12. J. Virol. 76, 11688-11703 (2002).
41. Kumar, A. & Lindner, V. Remodeling with neointima formation in the mouse carotid artery after cessation of blood flow. Arterioscler. Thromb. Vasc. Biol. 17, 2238-2244 (1997).
42. Sasieni, P. D. From genotypes to genes: doubling the sample size. Biometrics 53, 1253-1261 (1997).

1. A method of using of human or mice-ISG12 to develop drugs that modify the effect of ISG12 on the activity of transcription factors.

2. The method according to claim 1, where the transcription factor is a member of the nuclear receptor family.

3. The method according to claim 1, where the effect modification is achieved by interfering with the interaction of ISG12 with a specific transcription factor.

4. The method according to claim 1, where effect modification is achieved by interfering with the interaction of ISG12 with the nuclear export of the transcription factor.

5. The method according to claim 1, where the transcription factor is a member of the NR4A family.

6. The method according to claim 1, where the transcription factor is a member of the PPAR family.

7. The method according to claim 1, where the transcription factor is forming heterodimers with RXR.

8. The method according to claim 1 for preparing drugs for treatment of acute (e.g. sepsis) or chronic (e.g. rheumatic diseases, atherosclerosis) inflammatory diseases.

9. The method according to claim 1 for preparing drugs for treatment of metabolic (e.g. hyperglyceridemia, hypercholesterolemia, diabetes type II) diseases.

10. The method according to claim 1 for preparing drugs for treatment of vascular diseases.

11. The method according to claim 1 by using of ISG12-gene deficient animals.

12. The method according to claim 11 by using of ISG12-gene deficient animals for studies of inflammatory diseases.

13. The method according to claim 11 by using of ISG12-gene deficient animals for studies of metabolic diseases.

14. The method according to claim 11 by using of ISG12-gene deficient animals for studies of vascular diseases.

15. The method according to claim 11 by using of ISG12-gene deficient animals for studies for drugs for treatment of inflammatory diseases.

16. The method according to claim 11 by using of ISG12-gene deficient animals for studies for drugs for treatment of metabolic diseases.

17. The method according to claim 11 by using of ISG12-gene deficient animals for studies for drugs for treatment of vascular diseases.

18. A method of using of single nucleotide polymorphism in the ISG12-gene for diagnostic use.

19. The method according to claim 18 by using of single nucleotide polymorphism in the ISG12-gene to determine susceptibility for diseases.

20. The method according to claim 19 by using of single nucleotide polymorphism in the ISG12-gene to determine susceptibility for inflammatory diseases.

21. The method according to claim 19 by using of single nucleotide polymorphism in the ISG12-gene to determine susceptibility for metabolic diseases.

22. The method according to claim 19 by using of single nucleotide polymorphism in the ISG12-gene to determine susceptibility for vascular diseases.

23. The method according to claim 19 by using of single nucleotide polymorphism in the ISG12-gene to determine susceptibility for drugs.

24. The method according to claim 23 by using of single nucleotide polymorphism in the ISG12-gene to determine susceptibility for drugs for treatment of inflammatory diseases.

25. The method according to claim 23 by using of single nucleotide polymorphism in the ISG12-gene to determine susceptibility for drugs for treatment of metabolic diseases.

26. The method according to claim 23 by using of single nucleotide polymorphism in the ISG12-gene to determine susceptibility for drugs for treatment of vascular diseases.
